(19) United States
(12) Patent Application Publication
(10) Pub. No.: US 2011/0028785 A1
(43) Pub. Date: Feb. 3, 2011

(54) Endoscope with adjustable viewing angle

(75) Inventor: Yung-Hsiu Chen, San Chung (TW)

Correspondence Address: CHARLES E. BAXLEY, ESQUIRE, 90 JOHN STREET, SUITE 403, NEW YORK, NY, 10038, US

(73) Assignee: There In One Enterprises Co., Ltd.

(21) Appl. No.: 12/462,230

(22) Filed: Jul. 31, 2009

(51) Int. Cl. A61B 1/045; A61B 1/04

(52) U.S. Cl. 600/109; 600/173

(57) ABSTRACT

An endoscope with adjustable viewing angle has a viewing angle adjusting mechanism mounted on a handheld body and a control module mounted inside the handheld body. The viewing angle adjusting mechanism has a cable seat pivotally mounted on the handheld body, an adjustable knob mounted on the cable seat, a flexible tube, a hollow flexible cable joined with one end of a flexible tube, and two pull strings between the other end of the flexible tube and the adjusting knob. Given that, while the cable seat is rotated, the image acquisition module on one end of the flexible cable can be controlled to rotate synchronously. The relative pulling and releasing movement of the two pull strings driven by turning the adjusting knob makes the flexible tube bend correspondingly so as to further adjust the viewing angle of the image acquisition module.

### BACKGROUND OF THE INVENTION

1. Field of the Invention

The present invention relates to a portable endoscope, and more particularly to a magnification control to adjust a viewing angle.

2. Description of Related Art

Owing to developments of electronic technology, endoscopes have been extensively applied in various fields, such as national defense, medical science, industry, commerce, fishery, forestry, agriculture, education and so forth, among which medical applications prevail significantly. For example, Taiwan Utility Model Patent No. M309972 discloses an endoscope device and connection structure thereof With reference to FIG. 7, the endoscope device has a light source module (131), a viewing lens (132), a photo-sensing component (112), a circuit substrate (111), a flexible flat cable (133) and a connection module (113).

The light source module (131) emits at least a light beam to an outer environment. The viewing lens (132) collects a reflected light beam of the light beam to form an optical image. The photo-sensing component (112) generates an electronic signal in accordance with the optical image. The circuit substrate (111) is attached to the photo-sensing component (112) and receives the electronic signal. The flexible flat cable (133) transmits the electronic signal to a display device. The connection module (113) is vertically or horizontally connected to the circuit substrate (111) to electrically connect with the flexible flat cable (133) and serves as a signal transmission medium of the circuit substrate (111) and the flexible flat cable (133) for the flexible flat cable (133) to receive and transmit the electronic signal.

The endoscope includes a multitude of optical and electronic components for medical staff to internally view human bodies for endoscopic examination. Endoscopic examination is a very accurate and highly professional medical technique and the endoscope may be inserted via an esophagus to minimize impact and harm to a patient. As a result, endoscopic examination must be carefully conducted to achieve an accurate and definite examination. However, the prior art is unclear about providing reasonable support features. For example, the prior art only mentions that the signal transmission medium is flexible and bendable and when inserted into the human body, the viewing lens mounted on the end portion of the signal transmission medium can be controlled to move forward and backward by the medical staff. Therefore, convenience and efficiency of use of the endoscope is limited.

As such, the prior art fails to provide reasonable features, a structure of the endoscope needs to be further refined.

### SUMMARY OF THE INVENTION

The main objective of the present invention is to provide an endoscope with adjustable viewing angle, which allows an image acquisition module mounted on an end of a cable of an endoscope to change magnification to adjust a viewing angle after the cable of the endoscope is inserted into a human body, thereby facilitating detailed and complete endoscope examination.

To achieve the foregoing main objective, the endoscope with adjustable viewing angle has a handheld body, a control module, a viewing angle adjusting mechanism and an image acquisition module. The control module is mounted inside the handheld body to process and output an image. The viewing angle adjusting mechanism is mounted on the handheld body and has a cable seat, an adjusting knob, a flexible tube, a hollow flexible cable and two pull strings. The cable seat is pivotally mounted on the handheld body. The adjusting knob is mounted on the cable seat. The flexible tube has two ends. The hollow flexible cable has two ends. One end of the hollow flexible cable is securely mounted on the cable seat, and the other end thereof is joined with one end of the flexible tube. Each of the two pull strings has two ends. One ends of the two pull strings are fastened at opposite positions on the other end of the flexible tube, and other ends of the two pull strings are passed through the flexible tube and the flexible cable to couple with the adjusting knob on the cable seat.

The image acquisition module is mounted on the viewing angle adjusting mechanism for the viewing angle adjusting mechanism to adjust a viewing angle thereof and having a signal line passing through the viewing angle adjusting mechanism to connect with the control module inside the handheld body. When the cable seat on the handheld body is rotated, the image acquisition module is circularly rotated through the flexible cable to adjust the viewing angle; the two pull strings are further drawn and released relatively through the rotation of the adjustable knob on the cable seat so that the flexible tube bends in a corresponding direction to further adjust the viewing angle. As such, medical staff can conduct a thorough and complete endoscopic examination.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of an endoscope with adjustable viewing angle in accordance with the present invention;

FIG. 2 is a partial perspective view of a flexible tube in accordance with the present invention without a sleeve;

FIG. 3 is a front plane view of an image acquisition module in accordance with the present invention;

FIG. 4 is a partial cross-sectional view of the flexible tube in accordance with the present invention after the flexible tube is bent;

FIG. 5 is another cross-sectional view and another operational view of the flexible tube in accordance with the present invention;

FIG. 6 is a partial cross-sectional view of a cable seat in accordance with the present invention; and

FIG. 7 is an exploded perspective view of a conventional endoscope device and connection structure thereof.

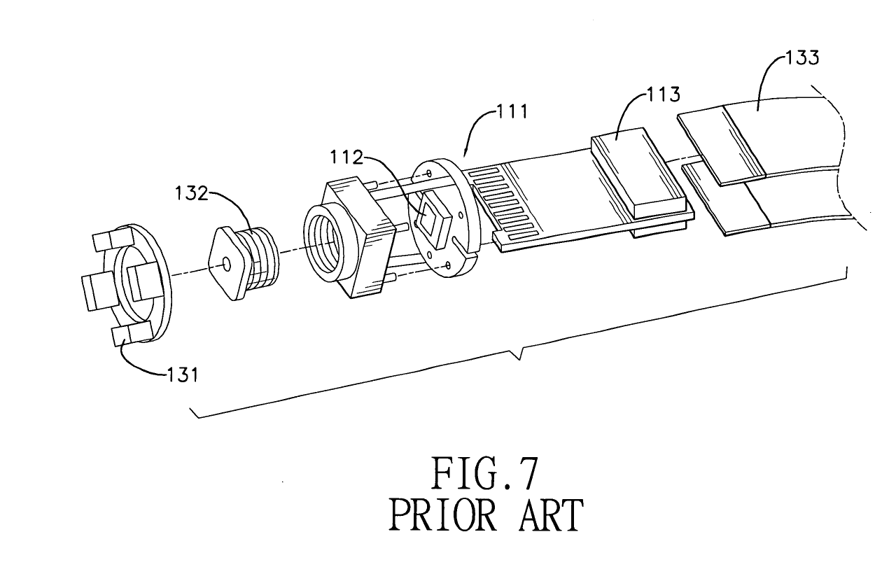

### DETAILED DESCRIPTION OF THE PREFERRED EMBODIMENT

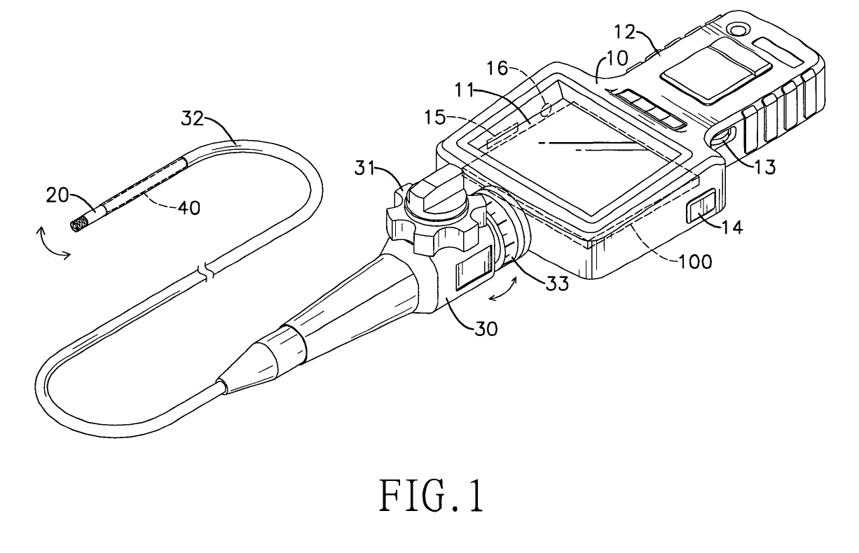

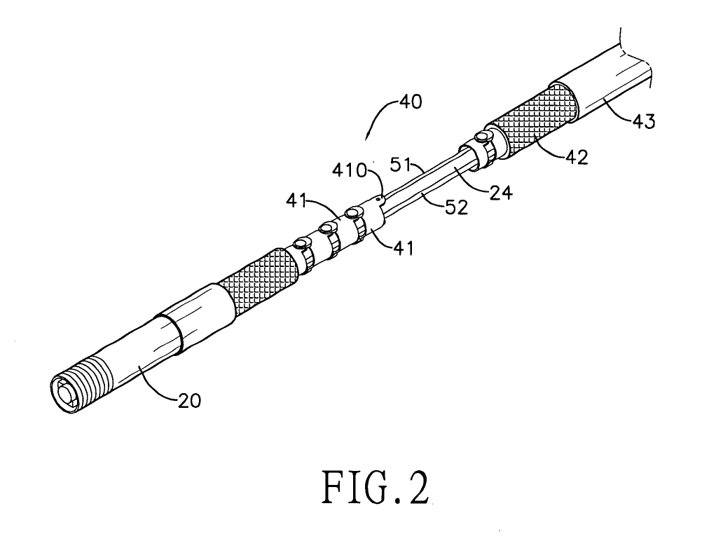

With reference to FIG. 1 and FIG. 2, the present invention has a handheld body (10), a viewing angle adjusting mechanism, a control module (100), and an image acquisition module (20).

The handheld body (10) has a wide end and a narrow end, a control module (100), a display device (11), a keyboard (12), a dial (13), a communication interface port (14), a memory card slot and a charging socket. The control module (100) is mounted inside the handheld body (10). The wide end serves as a handle for users to hold in operation. A window is formed on a surface with a larger area on the wide end of the handheld body (10). The display device (11) is mounted within the window and connected with an output end of the control module (100) to display the image signal after image processing transmitted from the image acquisition module (20) to the control module (100).

Moreover, the keyboard (12), the dial (13), the memory card slot and the charging socket are mounted on the narrow end of the handheld body (10) and the communication interface port (14) is mounted on the wide end of the handheld body (10) to facilitate operation and data transfer. In the present embodiment, the keyboard (12) is located on an top surface of the narrow end, the dial (13) and the communication interface port (14) are located on one side of the wide end, and the memory card slot (15) and the charging socket (16) are located on another side of the wide end. The aforementioned locations pertain to one among many feasible implementation solutions and are not limited to those as shown in the figure. Moreover, the communication interface port (14) may be a USB port.

The viewing angle adjusting mechanism has a cable seat (30), an adjustable knob (31), a hollow flexible cable (32), and a flexible tube (40) and two pull strings (51, 52). The viewing angle adjusting mechanism is mounted on the handheld body (10), the adjustable knob (31) is mounted on the cable seat (30) and the two pull strings.

The cable seat (30) is a hollow tube having a pivoting part (33) in the form of a hollow cylinder formed on one end thereof for pivotally mounting on the handheld body (10) so as to pivot in an opposite direction with respect to that of the handheld body (10). The pivoting part (33) has dials formed thereon for users to identify a degree of pivoting. The other end of the cable seat (30) tapers for joining to one end of the flexible cable (32). When the cable seat (30) pivots, the flexible cable (32) also coaxially pivots therewith.

The flexible tube (40) is inserted into the other end of the flexible cable (32) and received inside the flexible cable (32). The flexible tube (40) has two opposite ends and a length that is shorter than that of the flexible cable (32). One end of the flexible cable (40) is connected to the other end of the flexible cable (32). The image acquisition module (20) is mounted on the end of the flexible tube (40). A signal line (24) of the image acquisition module (20) passes through the flexible tube (40), the flexible cable (32) and the cable seat (30) to connect with the control module inside the handheld body (10).

The flexible tube (40) is composed of multiple bushings (41) which are pivotally connected and are respectively sheathed by a metal mesh layer (42) and a rubber sleeve (43) to serve as a protection. Furthermore, those bushings (41) are hollow and have two opening formed on two ends thereof respectively and have two pivoting ears (410) located oppositely and formed by respectively extending outwards from the two opening to be hinged with the respective pivoting ears of the neighboring bushing. Accordingly, each bushing (41) can be bent relative to its neighboring bushing. When the flexible tube is bent (40), the image acquisition module (20) located on one end thereof also changes its viewing direction. To facilitate bending and diverting the flexible tube (40), both one ends of the two pull strings (51), (52) are respectively fastened with two opposite positions on one end of the flexible tube (40) abutting the image acquisition module (20). Two pull strings (51), (52) pass through each of the bushings (41) of the flexible tube (40) to enter the flexible cable (32) and the cable seat (30) and then couple with an adjusting knob (31) pivotally mounted on the cable seat (30). The adjusting knob (31) is used to control the pull strings (51), (52).

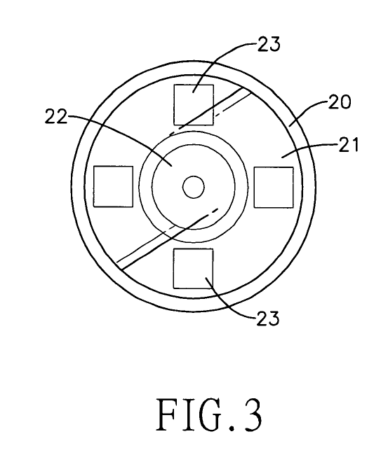

With further reference to FIG. 3, the image acquisition module (20) is mounted on the viewing angle adjusting mechanism and has a signal line (24), a circuit board (21), an image acquisition component (22) and a plurality of light emitting diodes (herein after LEDs) (23).

One end of the signal line (24) is electronically connected to the circuit board (21). The other end thereof passes through the flexible tube (40) to enter the flexible cable (32), the cable seat (30) and the handheld body (10) to electronically connect with the control module (not shown) mounted inside the handheld body (10). The image acquisition component (22) is electronically mounted on the circuit board (21) and may be a charge-coupled diode (CCD) or a CMOS. In the present embodiment, multiple LEDs are electronically mounted thereon for serving as a lighting source while recording images. The LEDs (23) are controlled by the control module, since the signal line (24) is electronically connected between the circuit board and the control module. Adjusting the dial (13) on the handheld body (10) controls a state and luminance of the LEDs (23).

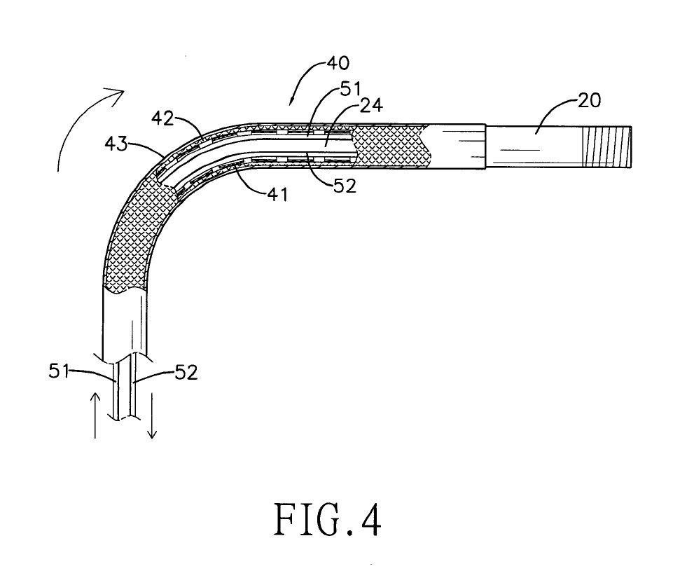

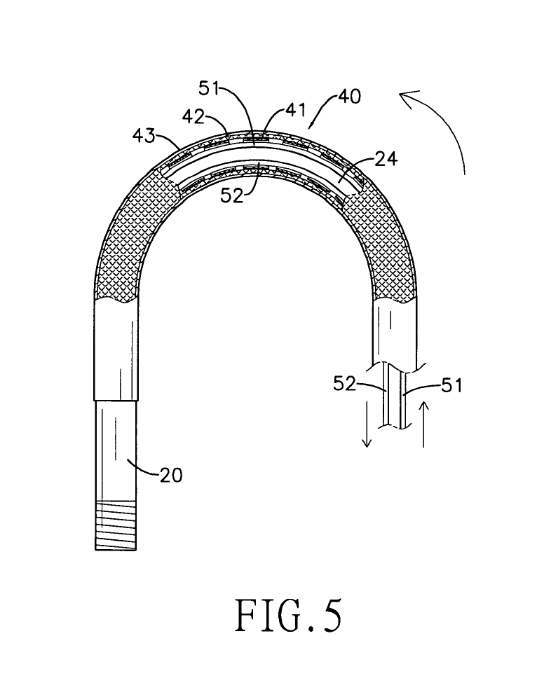

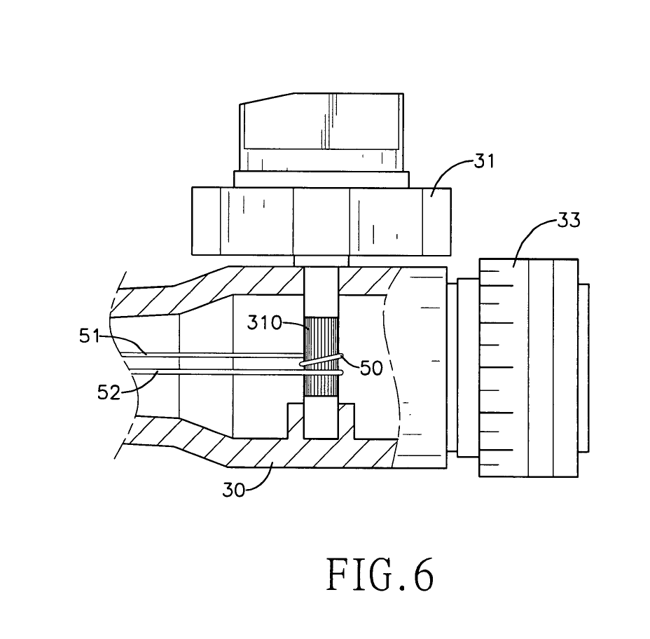

With reference to FIG. 4, when a pull string (51) is drawn (the direction shown in FIG. 4 is downward) and the other pull string (52) is relaxed, the flexible tube (40) will bend to the right as shown in FIG. 4. With reference to FIG. 5, when the pull string (52) is drawn and the other pull string (51) is relaxed, the flexible tube (40) will bend to the left, opposite to the direction as shown in FIG. 5. The curvature of the flexible tube (40) roughly makes the viewing direction of the image acquisition module (20) in parallel with the flexible cable (32). Therefore, the pull strings (51), (52) are drawn and released in turn, that is, one pull string (51) is drawn and the other pull string (52) is released, and the pull string (51) is released and the pull string (52) is drawn, in completion of the alternate drawing and releasing motion. With reference to FIG. 6, a linking wire (50) is wound around a knob shaft (310) of the adjusting knob (31) mounted on a cable seat (30) to generate an abrasive contact. The linking wire (50) is connected between the pull strings (51), (52). The linking wire (50) and the two pull strings (51), (52) may be a wire. Given that, when the adjusting knob (31) rotates clockwise or counterclockwise, the two pull strings (51), (52) are drawn and released respectively so that the flexible tube (40) bends in a corresponding direction, the curvature of the flexible tube (40) depends on the rotation range of the adjusting knob (31).

The foregoing description comprehensively described substantial structure of the present invention. The operation thereof is further depicted as follows.

When the flexible cable (32) of the endoscope passes through the esophagus to enter a patient, the flexible cable (32) is rotated through the rotation of the cable seat (30) and the image acquisition module (20) located on one end of the flexible cable (40) rotates with respect to an viewing direction to acquire a viewing angle ideal for visual observation. On the other hand, user can also draw and release the pull strings (51), (52) by turning the adjusting knob (31) on the cable seat (30) to bend the flexible tube (40) to change the viewing direction of the image acquisition module (20), thereby making the examination of the endoscope more thorough and complete.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. An endoscope with adjustable viewing angle, comprising:
a handheld body;
a control module mounted inside the handheld body to process and output an image;
a viewing angle adjusting mechanism mounted on the handheld body, having
a cable seat pivotally mounted on the handheld body;
an adjusting knob mounted on the cable seat;
a hollow flexible cable having two ends, one of two ends of the hollow flexible cable securely mounted on the cable seat;
a flexible tube inserted into the other end of the hollow flexible cable, received inside the hollow flexible cable and having two ends, wherein one end of the flexible tube is joined with the other end of the hollow flexible cable into which the flexible tube is inserted; and
two pull strings each of which has two ends, one ends of the two pull strings fastened at opposite positions on the end of the flexible tube joined with the other end of the hollow flexible cable, and other ends of the two pull strings passed through the flexible tube and the hollow flexible cable to couple with the adjusting knob on the cable seat; and
an image acquisition module mounted on the viewing angle adjusting mechanism for the viewing angle adjusting mechanism to adjust a viewing angle thereof and having a signal line passing through the viewing angle adjusting mechanism to electronically connect with the control module inside the handheld body.

2. The endoscope with adjustable viewing angle as claimed in claim 1, wherein
the flexible tube has a plurality of bushings pivotally connected to one another, the bushings are hollow and have an opening formed on each of two ends thereof, two pivoting ears located oppositely and formed by respectively extending outwards from the two opening to be hinged with the respective pivoting ears of the neighboring bushing; and
two pull strings respectively pass through each of the bushings of the flexible tube and the flexible cable to enter the cable seat so as to couple with the adjusting knob pivotally mounted on the cable seat.

3. The endoscope with adjustable viewing angle as claimed in claim 2, wherein the adjustable knob has:
a knob shaft pivotally mounted on the cable seat; and
a linking wire wound around the knob shaft to constitute an abrasive contact and connected between the pull strings.

4. The endoscope with adjustable viewing angle as claimed in claim 3, further comprising a metal mesh layer and a rubber sleeve that are sequentially sheathed on the bushings of the flexible tube.

5. The endoscope with adjustable viewing angle as claimed in claim 1, wherein the image acquisition module has:
a circuit board electronically the signal line;
an image acquisition component mounted on and electronically connected to the circuit board; and
a plurality of LEDs mounted on and electronically connected to the circuit board.

6. The endoscope with adjustable viewing angle as claimed in claim 2, wherein the image acquisition module has:
a circuit board electronically the signal line passing through the flexible tube to enter the flexible cable;
an image acquisition component mounted on and electronically connected to the circuit board; and
a plurality of LEDs mounted on and electronically connected to the circuit board.

7. The endoscope with adjustable viewing angle as claimed in claim 3, wherein the image acquisition module has:
a circuit board electronically the signal line;
an image acquisition component mounted on and electronically connected to the circuit board; and
a plurality of LEDs mounted on and electronically connected to the circuit board.

8. The endoscope with adjustable viewing angle as claimed in claim 4, wherein the image acquisition module has:
a circuit board electronically the signal line;
an image acquisition component mounted on and electronically connected to the circuit board; and
a plurality of LEDs mounted on and electronically connected to the circuit board.

9. The endoscope with adjustable viewing angle as claimed in claim 5, wherein the handheld body has:
a narrow end formed as a handle;
a wide end having an window;
a display mounted within the window and connected with an output end of the control module;
a communication interface port mounted on one side of the wide end; and
a keyboard, a dial, a memory card slot and a charging socket formed on the narrow end of the handheld body to connect with the control module.

10. The endoscope with adjustable viewing angle as claimed in claim 6, wherein the handheld body has:
a narrow end formed as a handle;
a wide end having an window;
a display mounted within the window and connected with an output end of the control module;
a communication interface port mounted on one side of the wide end; and
a keyboard, a dial, a memory card slot and a charging socket formed on the narrow end of the handheld body to connect with the control module.

11. The endoscope with adjustable viewing angle as claimed in claim 7, wherein the handheld body has:
a narrow end formed as a handle;
a wide end having an window;
a display mounted within the window and connected with an output end of the control module;
a communication interface port mounted on one side of the wide end; and
a keyboard, a dial, a memory card slot and a charging socket formed on the narrow end of the handheld body to connect with the control module.

12. The endoscope with adjustable viewing angle as claimed in claim 8, wherein the handheld body has:
a narrow end formed as a handle;
a wide end having an window;
a display mounted within the window and connected with an output end of the control module;
a communication interface port mounted on one side of the wide end;
a memory card slot and a charging socket mounted on the other side of the wide end; and
a keyboard, a dial formed on the narrow end of the handheld body to connect with the control module.
